(19) United States
(12) Patent Application Publication
(10) Pub. No.: US 2024/0315542 A1
(43) Pub. Date: Sep. 26, 2024

(54) SMART LIGHT ISOLATOR

(71) Applicant: SMITH & NEPHEW, INC., Memphis, TN (US)

(72) Inventors: York ZHAO, Andover, MA (US); Charles Mark SWEET, Newton, MA (US); BaoDuy TRAN, Andover, MA (US)

(73) Assignee: SMITH & NEPHEW, INC., Memphis, TN (US)

(21) Appl. No.: 18/603,851

(22) Filed: Mar. 13, 2024

Related U.S. Application Data
(60) Provisional application No. 63/491,140, filed on Mar. 20, 2023.

(51) Int. Cl. A61B 1/06; A61B 1/00; A61B 1/07; A61B 1/317; F21V 23/04; F21W 131/20

(52) U.S. Cl. CPC A61B 1/0655; A61B 1/00117; A61B 1/00126; A61B 1/0669; A61B 1/07; A61B 1/317; F21V 23/0457; G02B 6/0008; F21W 2131/20

(57) ABSTRACT

A method for operating a first light source of a surgical instrument includes selectively supplying from the first light source, illumination light to the surgical instrument via a light guide, supplying, from a light source-side of the light guide and separate from the illumination light, a safety light via the light guide, determining, at a computing device, whether the safety light is received at the light source-side of the light guide from an instrument-side of the light guide, and, in response to the determining, controlling the first light source to selectively deactivate or reduce an intensity of the illumination light.

### CROSS-REFERENCE TO RELATED APPLICATIONS

This application claims the benefit of U.S. Provisional Application No. 63/491,140, filed on Mar. 20, 2023 and titled “Smart Light Isolator.” The entire disclosure of the application referenced above is incorporated herein by reference.

### FIELD

The present disclosure relates to surgical instruments, and more particularly to endoscopes.

### BACKGROUND

During endoscopic surgical procedures, an endoscope (e.g., laparoscope, arthroscope) is provided light from a light source by way of a light guide. When an endoscope is not in use during the surgical procedure, the endoscope may be removed from the surgical site or the light guide may be un-plugged from the endoscope.

### SUMMARY

A method for operating a first light source of a surgical instrument includes selectively supplying from the first light source, illumination light to the surgical instrument via a light guide, supplying, from a light source-side of the light guide and separate from the illumination light, a safety light via the light guide, determining, at a computing device, whether the safety light is received at the light source-side of the light guide from an instrument-side of the light guide, and, in response to the determining, controlling the first light source to selectively deactivate or reduce an intensity of the illumination light.

In other features, methods according to the principles of the present disclosure are configured to perform one or more other functions as described herein.

A system for supplying illumination light to a surgical instrument includes a first light source, the first light source configured to supply the illumination light to the surgical instrument via a light port and a light guide coupled between the light port and the surgical instrument, a second light source configured to supply, from a light source-side of the light guide and separate from the illumination light, a safety light via the light guide, and a computing device configured to determine whether the safety light is received at the light source-side of the light guide from an instrument-side of the light guide, and in response to the determining, control the first light source to selectively deactivate or reduce an intensity of the illumination light.

In other features, systems or devices according to the principles of the present disclosure are configured to perform one or more other functions as described herein.

A surgical instrument includes a light post configured to couple the surgical instrument to a light guide to receive, from a light port, illumination light for illuminating a surgical site, and, separate from the illumination light, safety light, and an optical switch configured to receive the safety light and supply the safety light back to the light port via the light guide.

In other features, surgical instruments of the present disclosure one or more other structural features as described herein.

Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

For a detailed description of example embodiments, reference will now be made to the accompanying drawings in which:

FIG. 1 shows an example endoscopic system according to the present disclosure;

FIG. 2 shows a block diagram of an example endoscopic console according to the present disclosure;

FIG. 3 shows an exploded perspective view of the connectors of light guide according to the present disclosure;

FIG. 4 shows a cross-sectional view of the arthroscope-end connector coupled within the cable adaptor and the scope adaptor according to the present disclosure;

FIG. 5 shows an exploded perspective view of the connectors of another example light guide according to the present disclosure;

FIG. 6 shows a cross-sectional view of the arthroscope-end connector coupled within the scope adaptor according to the present disclosure;

FIG. 7 shows a partial transparent view turret or light port and an example turret connector according to the present disclosure;

FIG. 8 shows an exploded, perspective view of the connectors of the light guide according to the present disclosure;

FIG. 9 is a functional block diagram of an example system according to the present disclosure;

FIG. 10 shows a block diagram of an example computing device configured to implement functions of the systems and methods according to the present disclosure; and

FIG. 11 illustrates steps of an example method for controlling a light source of an endoscopic system according to the principles of the present disclosure.

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

“Controller,” “control circuitry,” or “computing device” shall mean, alone or in combination, individual circuit components, an application specific integrated circuit (ASIC), a microcontroller with controlling software, a reduced-instruction-set computing (RISC) with controlling software, a digital signal processor (DSP), a processor with controlling software, a programmable logic device (PLD), a field programmable gate array (FPGA), or a programmable system-on-a-chip (PSOC), configured to read inputs and drive outputs responsive to the inputs.

“A”, “an”, and “the” as used herein refers to both singular and plural referents unless the context clearly dictates otherwise. By way of example, “a processor” programmed to perform various functions refers to one processor programmed to perform each and every function, or more than one processor collectively programmed to perform each of the various functions.

### DETAILED DESCRIPTION

The following discussion is directed to various embodiments of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

During endoscopic surgical procedures, an endoscope (e.g., laparoscope, arthroscope) is provided light from a light source by way of a light guide. When an endoscope is not in use during the surgical procedure, the endoscope may be removed from the surgical site or the light guide may be un-plugged from the endoscope. When the light guide is unplugged from the endoscope, high-intensity light may shine into the surgical room from the end of the light guide. The high-intensity light may cause thermal and over-illumination hazards.

Systems and methods of the present disclosure are configured to controlling the light output (e.g., turning the light output on and off, reducing or increasing intensity of the light output, etc.) in response to control signals, inputs, sensed conditions, etc. Various examples are directed to limiting exposure to light associated with the endoscope. More particularly, examples are directed to determining, by an endoscopic console, that the light guide has been disconnected from the endoscope, and, in response to the determining, reducing or turning off the light output from the endoscopic console. More particularly still, example systems utilize an optical fiber, separate and distinct from the main light path, which senses when the light guide is connected and disconnected. When the light guide is disconnected, illumination light from the endoscopic console is reduced, in some cases reduced to zero (e.g., turned off).

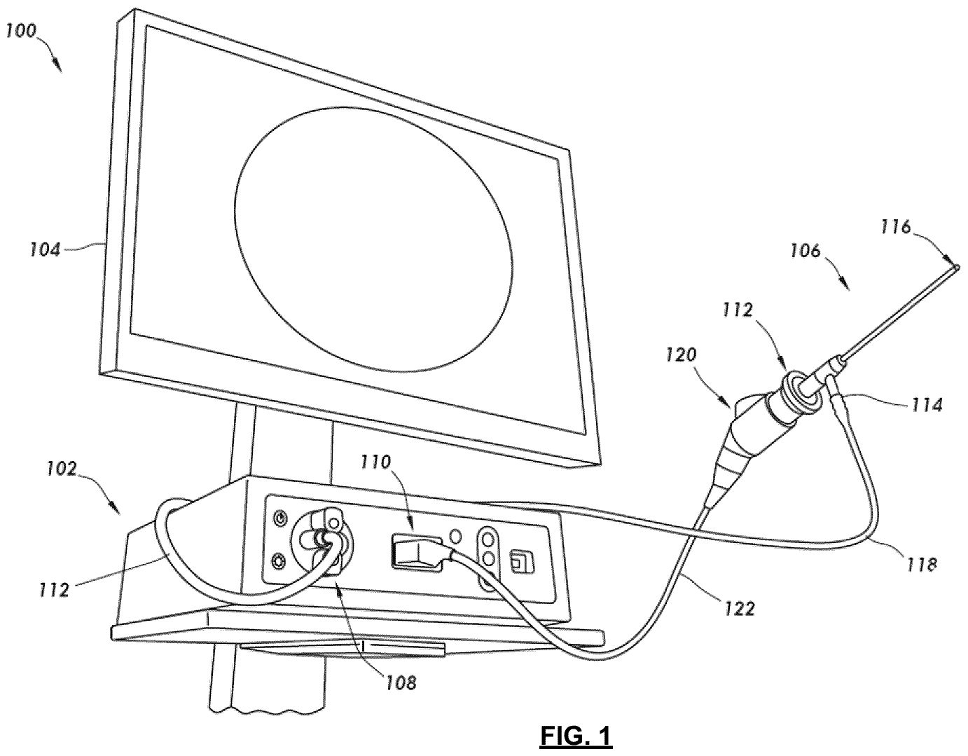

FIG. 1 shows an endoscopic system in accordance with at least some embodiments of the present disclosure. In particular, FIG. 1 shows an endoscopic system 100 comprising an endoscopic console 102 coupled to a display device 104 and coupled to an endoscope (which is shown as, and may be referred to as, an arthroscope 106). The endoscopic console 102 includes a light port 108 and a camera port 110. Each of the ports 108 and 110 is disposed on/arranged in an outside surface of the endoscopic console 102. The example arthroscope 106 includes a camera-head connector 112 (on a proximal end of the arthroscope 106), a light post 114, and a distal end 116. Light received via the light port 108 and a light guide 118 shines out of the distal end 116 and reflected light propagates back into the arthroscope 106 through the distal end 116. A camera head 120 is mechanically and optically coupled to the camera-head connector 112 such that an optical array within the camera head 120 captures images of tissue and structures beyond the distal end 116 of the arthroscope 106. The example camera head 120 is electrically coupled to the camera port 110 by way of an electrical cable 122, though any suitable communicative connection between the camera head 120 and the endoscopic console 102 may be used. The endoscopic console 102 may thus receive individual electronic images, or streams of electronics images in the form of video images, and display the images on the display device 104.

The light guide 118 is optically coupled between the light port 108 on the endoscopic console 102 and the light post 114 on the arthroscope 106. In some examples, the light guide 118 may include one or more optical fibers configured and constructed to carry light from a light source within the endoscopic console 102 to the light port 108.

The endoscopic console 102 according to the present disclosure is configured to control the light output (e.g., from the light port 108) in response to, for example, the light guide 118 being disconnected from the endoscopic console 102. For example, the endoscopic console 102 is configured to determine whether the light guide 118 is disconnected from the light port 108 and, in response to determining that the light guide 118 is disconnected from the light port 108, reducing or turning off the light output from the endoscopic console 102. In examples, the endoscopic console 102 includes one or more computing devices, processors or processing devices, etc. configured to control the light output from the light port 108 as described below in more detail.

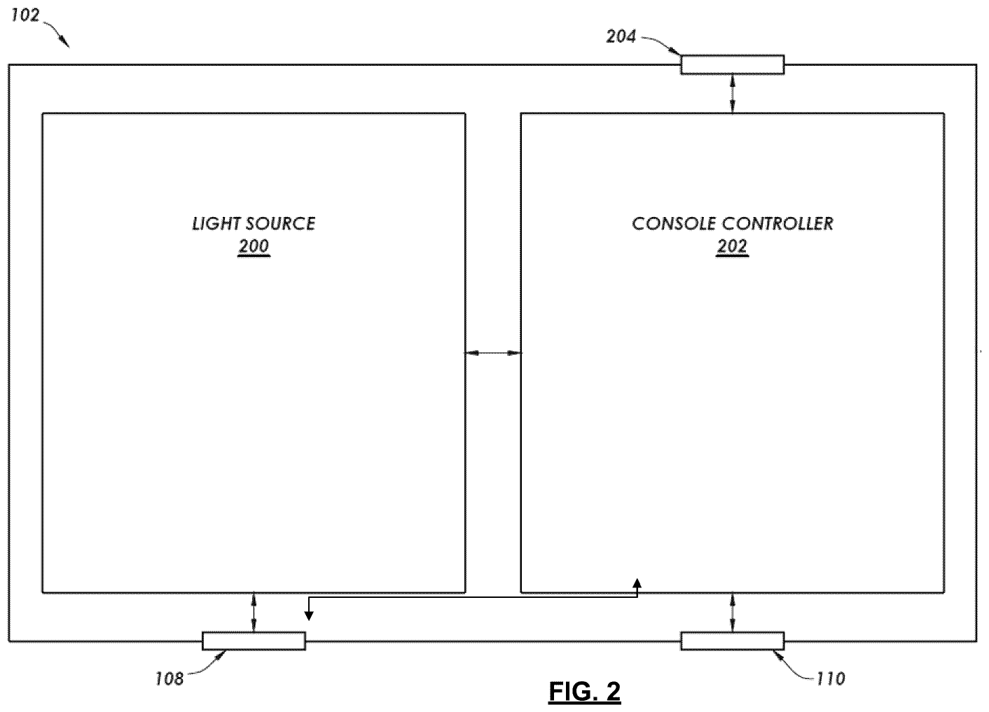

FIG. 2 shows a block diagram of the endoscopic console 102 in accordance with at least some embodiments of the present disclosure. In particular, as shown in FIG. 2, the endoscopic console 102 comprises the light port 108 and the camera port 110, each exposed on an outer surface of the endoscopic console 102. Additionally, the example endoscopic console 102 defines a display port 204 arranged and exposed on the outer surface of the endoscopic console 102. The display port 204 is configured and constructed to operatively couple to a display device, such as the display device 104 (as shown in FIG. 1). The example endoscopic console 102 includes (e.g., enclosed within a housing/outer surface of the console 102) a light source 200 and a console controller 202. For example, the console controller 202 includes one or more computing devices, processors, etc. configured to perform one or more functions of the systems and methods of the present disclosure as described below in more detail.

The example light source 200 may have various example implementations. In some examples, the light source 200 may be an incandescent bulb in combination with a moveable screen assembly with a varying width aperture. The intensity or illumination level of the light provided to the light port 108 from the light source 200 may be controlled by controlling (e.g., using the console controller 202 or other control circuitry) the voltage provided to the incandescent bulb, controlling a position of the moveable screen, or both. In yet still other cases, the light source may be a xenon-based florescent bulb, again possibly in combination with a moveable screen assembly. The intensity of the light provided to the light port 108 from the light source 200 when using a Xenon-based fluorescent bulb may be controlled by controlling (e.g., using the console controller 202 or other control circuitry) the electrical energy (e.g., voltage, current, or both) provided to the xenon gas within the florescent bulb, controlling the position of the moveable screen, or both. In yet still other cases, the light source 200 may be a laser-light source, again possibly in combination with a moveable screen assembly. The intensity of the light provided to the light port 108 from the light source 200 when using a laser-light source may be controlled by controlling (e.g., using the console controller 202 or other control circuitry) the electrical energy (e.g., voltage, current, or both) provided to the laser-light source, controlling the position of the moveable screen, or both. In yet still other cases, the light source 200 may be a series of light-emitting diodes (LEDs). The intensity of the light provided to the light port 108 from the light source 200 when using LEDs may be controlled by controlling (e.g., using the console controller 202 or other control circuitry) an average current through the LEDs. In summary, any suitable light source whose light intensity may be controlled electronically or mechanically may be used for the light source 200.

The console controller 202 is coupled to the light source 200, the camera port 110, the display port 204, and the light port 108. In one example, the console controller 202 may be an application specific integrated circuit (ASIC) configured and constructed to perform the various methods discussed herein. In other cases, the console controller 202 may be a microcontroller with controlling software, along with various input devices and output devices, configured and constructed to implement the various systems and methods discussed herein. In further cases, the console controller 202 may be a processor, such as reduced-instruction-set computing (RISC), a digital signal processor (DSP), or a general purpose processor, along with controlling software configured and constructed to implement the various systems and methods discussed herein. Further still, the console controller 202 may be a programmable logic device (PLD) or a field programmable gate array (FPGA) configured and constructed to implement the various systems and methods described herein. Yet further still, the console controller 202 may be implemented as combinations of any of the recited implementations.

In example systems, the console controller 202 is coupled to the light port 108 to implement the smart light features of reducing or turning off the light from the light source 200 when the arthroscope 106 is disconnected from the light guide 118.

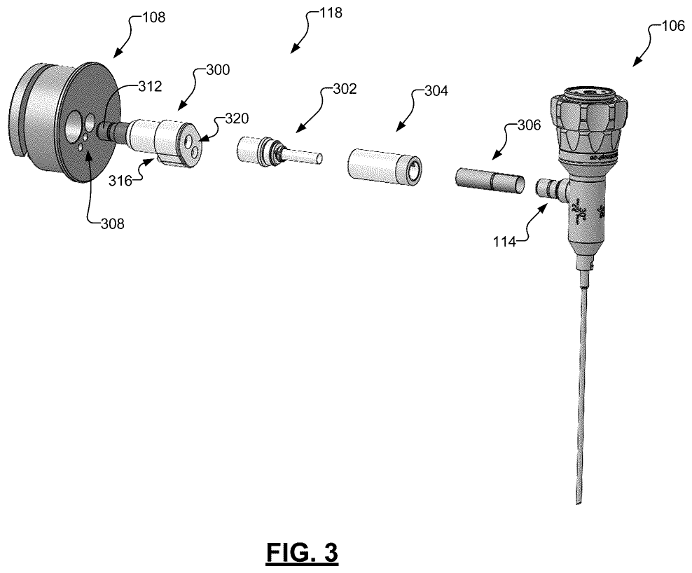

FIGS. 3-8 illustrate example implementations of various components of an arthroscopic assembly (e.g., the arthroscope 106 and various components connected to the arthroscope 106) according to the principles of the present disclosure. For example, FIG. 3 shows an exploded perspective view of the connectors of the light guide 118 in accordance with at least some embodiments. In particular, FIG. 3 illustrates the light port (or turret) 108, as well as the arthroscope 106 and associated light post 114. Various connectors that optically couple the arthroscope 106 to the light source 200 within the endoscopic console 102 are arranged between the light port 108 and the light post 114. In particular, arranged on a proximal end of the light guide 118 is a turret connector 300 of the light guide 118, and arranged on a distal end of the light guide 118 is an arthroscope-end connector 302. Cables including the actual optical channels that extend between the turret connector 300 and the arthroscope-end connector 302 are not shown in FIG. 3. Also shown are a cable adaptor 304 and a scope adaptor 306 configured to couple the light guide 118 to the light post 114 of the arthroscope 106.

The turret connector 300 is configured to physically couple to the light port 108, and thus optically couples to the light source 200 within the endoscopic console 102. As discussed below in more detail, physically coupling the turret connector 300 also optically couples optical safety fibers extending from the turret connector 300 on the proximal end of the light guide 118 to the to the arthroscope-end connector 302 on the distal end of the light guide 118. The optical safety fibers are distinct from light channels, within cabling of the light guide 118, configured to carry light used for illumination (i.e., to carry light for output from the distal end 116 of the arthroscope), though the light channels within the light guide 118 may themselves be optical fibers. When the arthroscopic-end connector 302 is coupled to the cable adaptor 304, the scope adaptor 306, and the light post 114, optical signals carried along a first optical safety fiber are directed to return along a second optical safety fiber to the turret or light port 108. In other words, the first and second optical safety fibers of the light guide 118 are configured to carry optical signals in a first direction from the light port 108 toward the arthroscope 106 and in a second direction from the arthroscope 106 back toward the light port 108.

In an example, the light port 108 includes one or more optical signal ports or openings 308 configured to pass light from the light source 200, through the light port 108, and into the light guide 118 (e.g., via the turret connector 300). The turret connector 300 includes one or more optical signal insertion portions configured to couple with (e.g., insert into) respective optical signal openings 308. For example, the turret connector 300 includes a first (e.g., illumination optical signal) insertion portion 312 and a second (e.g., safety optical signal) insertion portion (e.g., corresponding to an optical fiber connector 316, only partially shown in FIG. 3). In this manner, light from the light source 200 passes through the openings 308 and into respective light channels (e.g., optical fibers) via the insertion portion 312 and the optical fiber connector 316. The light channels are routed through respective openings of various components of the light guide (e.g., openings 320 in the turret connector 300, the arthroscopic-end connector 302, the cable adaptor 304, and the scope adaptor 306) from the light port 108 to the light post 114. Although shown with the openings 308 disposed on the light port and the insertion portion 312 and the optical fiber connector 316 disposed on the turret connector 300, respectively, in other implementations the light port 108 may include insertion portions/connectors while the turret connector 300 includes openings configured to receive insertion portions/connectors of the light port 108.

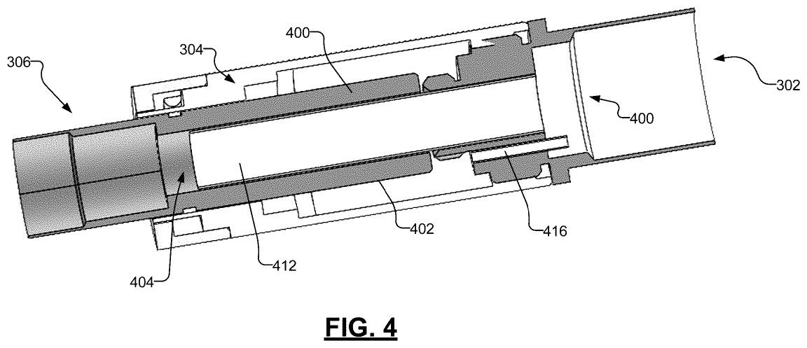

FIG. 4 shows a cross-sectional view of a connector assembly including the arthroscope-end connector 302, the cable adaptor 304, and the scope adaptor 306. For example, as shown, the arthroscope-end connector 302 is coupled to the scope adapter 306 and the arthroscope-end connector 302 and the scope adapter 306 are partially enclosed within the cable adaptor 304. For example, the scope adaptor 306 defines a cylinder 400 with an external diameter 402 and an internal volume 404. In an example, the cable adaptor 304 is configured and constructed to telescope over the cylinder 400 and provide a quick release connection to the scope adaptor 306. The arthroscope-end connector 302 defines an insertion portion 412 that defines an interior volume or channel that encloses the optical elements (e.g., optical fibers) configured to carry the illumination light. When the arthroscope-end connector 302 is coupled to the cable adaptor 304 and the scope adaptor 306, the optical elements configured to carry the illumination light abut the distal end of the light post 114 (not shown in FIG. 4) of the arthroscope 106.

Also shown in FIG. 4 are one or more optical safety fibers 416 configured to carry light used for optical safety functions (e.g., smart light functions) of the present disclosure. In particular, the optical safety fibers 416 extend along (e.g., within) the light guide 118 from the light port 108, through the turret connector 300, and to the arthroscope-end connector 302 and are coupled to the arthroscope-end connector 302. Distal ends of the optical safety fibers 416 are associated with an optical switch, reflector, or other component (not shown in FIG. 4) such that, when the light guide 118 is properly coupled to the light post 114 of the arthroscope 106, the optical switch is configured and constructed to transfer light carried along a first optical safety fiber to the light post 114 and back towards the endoscopic console 102 along a second optical safety fiber. In other examples, a single optical safety fiber may be used to implement the smart light functions. In these examples, the optical signal may be carried to the distal end by the optical safety fiber and, when the light guide 118 is properly coupled between the light port 108 and the light post 114, the light may be reflected and travel back toward the light port 108.

In an example, the optical switch is located within the light post 114. In other examples, the optical switch is located within the arthroscope 106. In still other examples, the optical switch is located within a component of the light guide 118 (e.g., the arthroscope-end connector 302, the cable adaptor 304, the scope adaptor 306, etc.). The arthroscope assembly may include more than one of the optical switches. In various examples, transmission of light from the light port 108 to the light post 114 and back to the light port 108 may be interrupted in response to any components of the light guide 118 being disconnected or uncoupled.

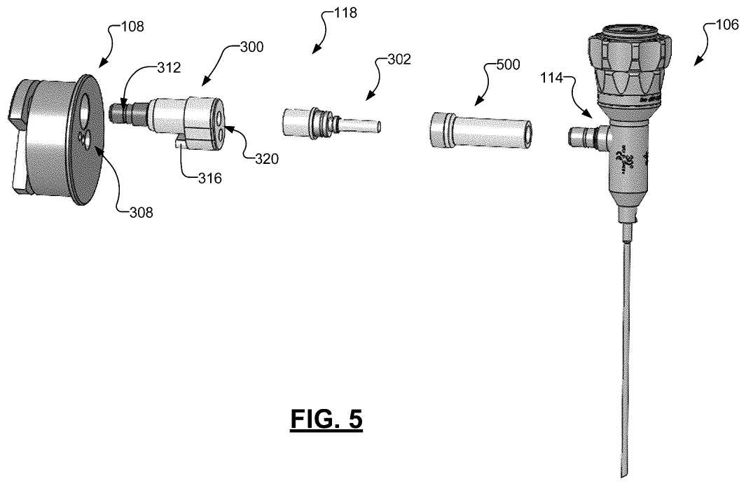

FIG. 5 shows an exploded perspective view of the connectors of another example implementation of the light guide 118 according to the present disclosure. In particular, FIG. 5 again shows the turret or light port 108, the arthroscope 106, and the light post 114. Similar to the example shown in FIG. 3, various connectors are arranged between the light port 108 and the light post 114 to optically couple the arthroscope 106 to the light source 200 within the endoscopic console 102, including the turret connector 300 of the light guide 118 and the arthroscope-end connector 302. Also shown in FIG. 5 is a scope adaptor 500, which replaces the cable adaptor 304 and the scope adaptor 306 of FIG. 3. In other words, in this example, the single scope adaptor 500 is configured to couple the light guide 118 to the light post 114 of the arthroscope 106 (i.e., rather than the two-connector configuration of the cable adaptor 304 and the scope adaptor 306 as shown FIG. 3).

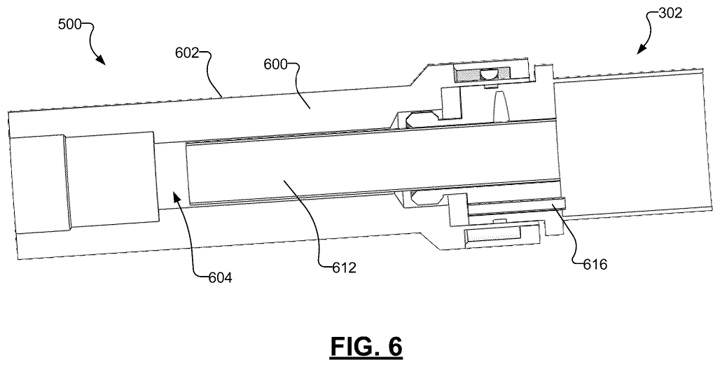

FIG. 6 shows a cross-sectional view of the arthroscope-end connector 302 coupled to the scope adaptor 500. In this example, the scope adaptor 500 couples directly the light post 114 (i.e., without an intervening cable adaptor coupled between the scope adaptor 500 and the light post 114). The scope adaptor 500 defines a cylinder 600 with an external diameter 602 and an internal volume 604. The arthroscope-end connector 302 is configured and constructed to telescope into the interior volume 604 of the cylinder 600. The scope adaptor 500 provides a quick release connection to the arthroscope-end connector 302. The arthroscope-end connector 302 defines an insertion portion 612 (e.g., corresponding to the insertion portion 412 of FIG. 4) that defines an interior volume or channel that encloses the optical elements (e.g., optical fibers) configured to carry the illumination light. When the arthroscope-end connector 302 is coupled to the scope adaptor 500, the optical elements configured to carry the illumination light abut the distal end of the light post 114 (not shown in FIG. 6) of the arthroscope 106.

Also shown in FIG. 6 are one or more optical safety fibers 616 configured to carry light used for optical safety functions (e.g., smart light functions) of the present disclosure. In particular, the optical safety fibers 616 extend along (e.g., within) the light guide 118 from the light port 108, through the turret connector 300, and to the arthroscope-end connector 302 and are coupled to the arthroscope-end connector 302. Distal ends of the optical safety fibers 616 are associated with an optical switch, reflector, or other component (not shown in FIG. 6) as described above in FIG. 4. Accordingly, when the light guide 118 is properly coupled to the light post 114 of the arthroscope 106, the optical switch is configured and constructed to transfer light carried along a first optical safety fiber to the light post 114 and back towards the endoscopic console 102 along a second optical safety fiber. In other examples, a single optical safety fiber may be used to implement the smart light functions. In these examples, the optical signal may be carried to the distal end by the optical safety fiber and, when the light guide 118 is properly coupled between the light port 108 and the light post 114, the light may be reflected and travel back toward the light port 108.

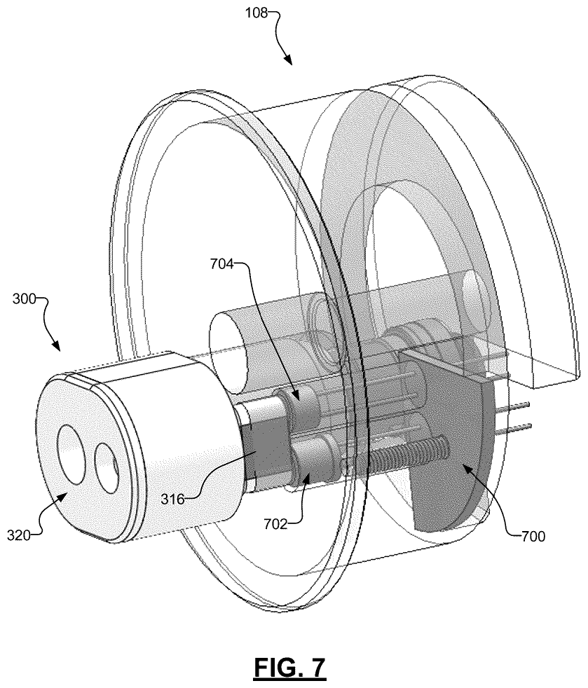

FIG. 7 shows a partial transparent view of the turret or light port 108 and the turret connector 300. As shown, the turret connector 300 is mechanically and optically coupled to the light port 108. Disposed within the light port 108 is example circuitry (e.g., a printed circuit board, or PCB) 700 coupled to a light source, such as a light-emitting diode (LED) 702, and an optical sensor or receiver 704. The LED 702 is configured and constructed to optically couple to a first safety fiber in the fiber connector 316. Optical signals generated by the LED 702 propagate toward the distal end of the light guide 118 in the first optical safety fiber. When the light guide 118 is properly coupled to the light post 114 of the arthroscope 106, an optical switch as described herein transfers light carried on the first optical safety fiber to the second optical safety fiber. The optical signal is carried, via the second optical safety fiber (or, in some examples via the first optical safety fiber), back to the optical receiver 704, which is optically coupled to the second optical safety fiber. Accordingly, the LED 702, the optical switch, and the optical receiver 704 form a complete optical circuit when the light guide 118 is coupled to both the light port 108 and the light post 114 and the connectors of the light guide 118 are properly coupled together. When the optical circuit is complete, the console controller 202 is configured and constructed to enable (e.g., activate or turn on) the light source 200. Conversely, when the optical circuit is broken (e.g., by disconnecting the light guide 118 from the light post 114 of the arthroscope 106 or disconnecting any of the connectors of the light guide 118 from each other), the console controller 202 is configured and constructed to disable/deactivate the light source 200, to command the light source 200 to significantly reduce the light provided to the light guide 118, etc.

As an example, the console controller 202 is configured to control the light source 200 to provide light to the light port 108 in response to user commands or inputs, in accordance with a control program, in accordance with a surgical procedure being performed, etc. During periods where the light source is supplying light, the LED 702 is activated (e.g., by the console controller 202) to supply light to the first optical safety fiber. Conversely, the optical receiver 704 is configured to generate, and provide to the console controller 202 (e.g., via the circuitry 700), an output indicative of whether the optical receiver 704 is receiving light (e.g., in a simplest example implementation, a binary signal, such as respective voltages indicative of a binary 1 or 0, a nonzero voltage or a zero voltage, etc.). The console controller 202 is configured to disable/turn off and/or reduce the intensity of the light source 200 in response to the output of the optical receiver 704 indicating that the optical receiver 704 is not receiving light back from the optical switch. Although as shown the LED 702 and the optical receiver 704 are located within the light port 108, in other examples the LED 702 and the optical receiver 704 may be located within other components, such as within a component of the light guide 118, within the endoscopic system 100, etc.

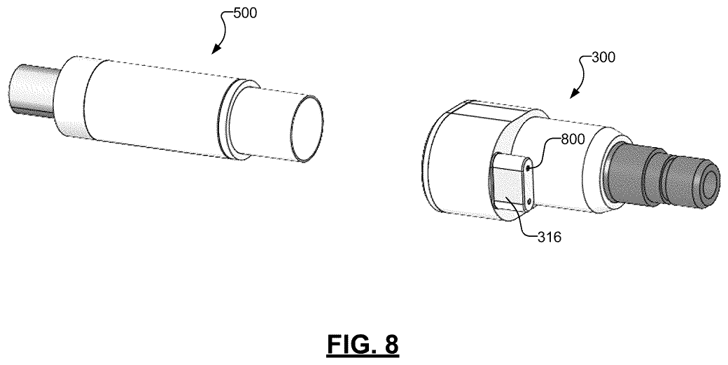

FIG. 8 shows an exploded, perspective view of the turret connector 300 and the scope adaptor 500. In this example, ports or openings 800 in the fiber connector 316 for respective optical safety fibers are shown. The openings 800 provide channels for the optical safety fibers from the LED 702 into the light guide 118 and back to the optical receiver 704.

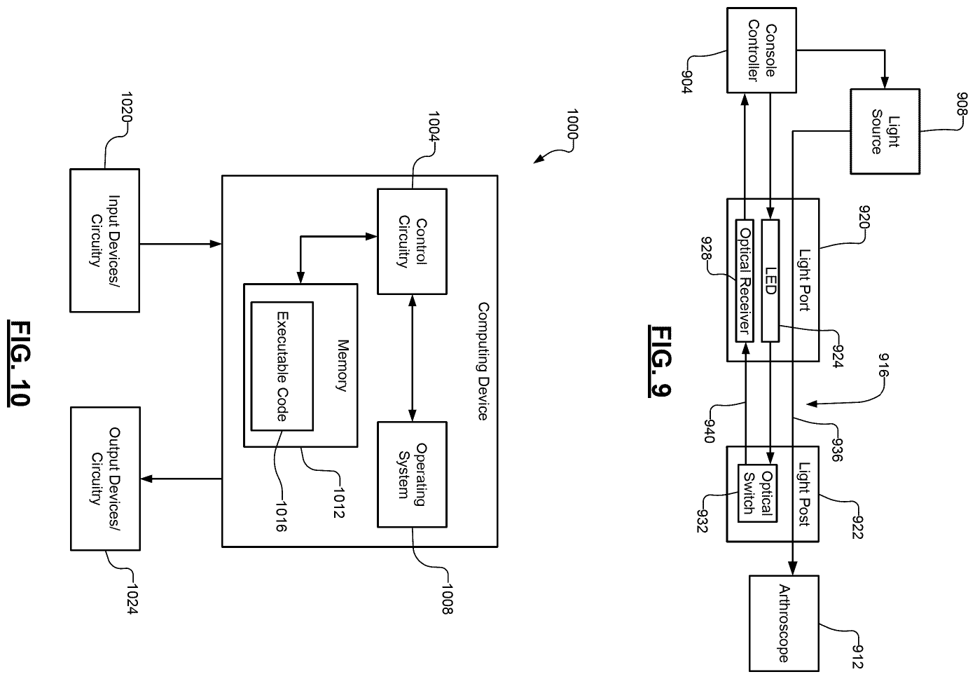

FIG. 9 is a functional block diagram of an example system 900 according to the principles of the present disclosure. The system 900 includes a console controller 904 configured to control a light source 908 as described above. For example, the console controller 904 selectively activates and deactivates the light source 908, adjust an intensity of light provided by the light source 908, etc. The light source 908, responsive to the console controller 904, supplies light to an arthroscope 912 (e.g., via a light guide 916). Various connectors of the light guide 916 as described herein are omitted from FIG. 9 for simplicity.

The light guide 916 is coupled between a light port 920 (e.g., of an endoscopic system) and a light post 922 (e.g., of the arthroscope 912). The light port 920 includes an LED 924 and optical receiver 928 as described above in FIG. 7. The light post 922 includes an optical switch 932 as described herein. Accordingly, the LED 924 is configured to supply light to the optical switch 932 (e.g., via a first optical safety fiber 936 of the light guide 916). The optical switch 932 is configured to receive the light supplied from the LED 924 via the first optical safety fiber 936 and supply light back to the optical receiver 928 (e.g., via a second optical safety fiber 940 of the light guide 916). The console controller 904 is responsive to an output of the optical receiver 928 that indicates whether the optical receiver 928 is receiving light via the second optical safety fiber 940 as described herein.

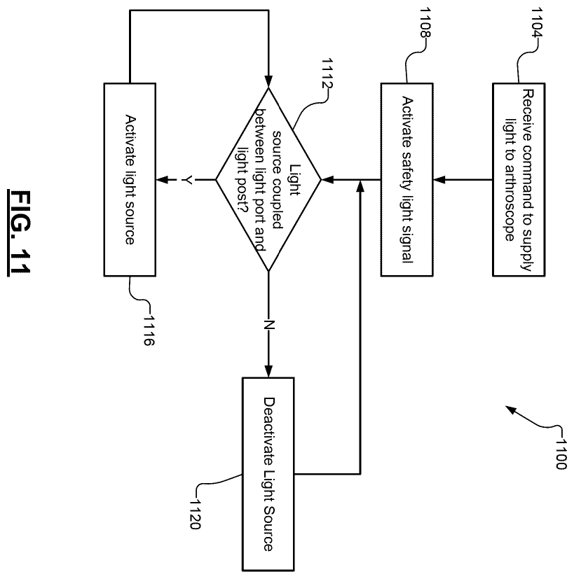

FIG. 10 shows a block diagram of an example computing device 1000 configured to implement functions of the systems and methods described herein according to the present disclosure. For example, one or more of the computing devices 1000 may implement or be implemented by the one or more components of the console controller 904. Systems described herein may implement a single computing device, a plurality of computing devices, etc., configured to individually and/or collectively perform functions related to the systems and methods of the present disclosure.

The computing device 1000 may include control circuitry 1004 that may be, for example, one or more processors or processing devices, a central processing unit processor, an integrated circuit or any suitable computing or computational device, an operating system 1008, a memory 1012, executable code 1016, input devices or circuitry 1020, and output devices or circuitry 1024. The control circuitry 1004 (or one or more controllers or processors, possibly across multiple units or devices) may be configured to implement functions of the systems and methods described herein. More than one of the computing devices 1000 may be included in, and one or more of the computing devices 1000 may act as the components of, a system according to embodiments of the disclosure. Various components of the computing device 1000 may be implemented with same or different circuitry, same or different processors or processing devices, etc.

The operating system 1008 may be or may include any code segment (e.g., one similar to the executable code 1016 described herein) configured and/or configured to perform tasks involving coordination, scheduling, arbitration, supervising, controlling or otherwise managing operation of the control circuitry 1004 (e.g., scheduling execution of software programs or tasks or enabling software programs or other hardware modules or units to communicate). The operating system 1008 may be a commercial operating system. The operating system 1008 may be an optional component (e.g., in some embodiments, a system may include a computing device that does not require or include the operating system 1008). For example, a computer system may be, or may include, a microcontroller, an application specific circuit (ASIC), a field programmable array (FPGA), network controller (e.g., CAN bus controller), associated transceiver, system on a chip (SOC), and/or any combination thereof that may be used without an operating system.

The memory 1012 may be or may include, for example, Random Access Memory (RAM), read only memory (ROM), Dynamic RAM (DRAM), Synchronous DRAM (SD-RAM), a double data rate (DDR) memory chip, Flash memory, volatile memory, non-volatile memory, cache memory, a buffer, a short-term memory unit, a long-term memory unit, or other suitable memory units or storage units. The memory 1012 may be or may include a plurality of memory units, which may correspond to same or different types of memory or memory circuitry. The memory 1012 may be a computer or processor non-transitory readable medium, or a computer non-transitory storage medium, e.g., RAM.

The executable code 1016 may be any executable code, e.g., an application, a program, a process, task, or script. The executable code 1016 may be executed by the control circuitry 1004, possibly under control of the operating system 1008. Although, for the sake of clarity, a single item of the executable code 1016 is shown, a system according to some embodiments of the disclosure may include a plurality of executable code segments similar to the executable code 1016 that may be loaded into the memory 1012 and cause the control circuitry 1004 to carry out methods described herein. Where applicable, the terms “process” and “executable code” may be used interchangeably herein. For example, verification, validation and/or authentication of a process may mean verification, validation and/or authentication of executable code.

In some examples, the memory 1012 may include non-volatile memory having the storage capacity of a storage system. In other examples, the computing device 1000 may include or communicate with a storage system and/or database. Such a storage system may include, for example, flash memory, memory that is internal to, or embedded in, a micro controller or chip, a hard disk drive, a solid-state drive, a CD-Recordable (CD-R) drive, a Blu-ray disk (BD), a universal serial bus (USB) device or other suitable removable and/or fixed storage unit. Content may be stored in the storage system and loaded from the storage system into the memory 1012 where it may be processed by the control circuitry 1004.

The input circuitry 1020 may be or may include any suitable input devices, components, or systems, e.g., physical sensors such as accelerometers, thermometers, microphones, analog to digital converters, etc., a detachable keyboard or keypad, a mouse, etc. The output circuitry 1024 may include one or more (possibly detachable) displays or monitors, motors, servo motors, speakers and/or any other suitable output devices. Any applicable input/output (I/O) devices may be connected to the control circuitry 1004. For example, a wired or wireless network interface card (NIC), a universal serial bus (USB) device, or external storage device may be included in the input circuitry 1020 and/or the output circuitry 1024. It will be recognized that any suitable number of input devices and output devices may be operatively connected to the control circuitry 1004. For example, the input circuitry 1020 and the output circuitry 1024 may be used by a technician or engineer in order to connect to the control circuitry 1004, update software, and the like.

Embodiments may include an article such as a computer or processor non-transitory readable medium, or a computer or processor non-transitory storage medium, such as for example memory, a disk drive, or USB flash memory, encoding, including or storing instructions (e.g., computer-executable instructions, which, when executed by a processor or controller, carry out methods disclosed herein), a storage medium such as the memory 1012, computer-executable instructions such as the executable code 1016, and a controller such as the control circuitry 1004.

The storage medium may include, but is not limited to, any type of disk including magneto-optical disks, semiconductor devices such as read-only memories (ROMs), random access memories (RAMs), such as a dynamic RAM (DRAM), erasable programmable read-only memories (EPROMs), flash memories, electrically erasable programmable read-only memories (EEPROMs), magnetic or optical cards, or any type of media suitable for storing electronic instructions, including programmable storage devices.

Embodiments of the disclosure may include components such as, but not limited to, a plurality of central processing units (CPU) or any other suitable multi-purpose or specific processors or controllers (e.g., controllers similar to the control circuitry 1004), a plurality of input units, a plurality of output units, a plurality of memory units, and a plurality of storage units, etc. A system may additionally include other suitable hardware components and/or software components. In some embodiments, a system may include or may be, for example, a personal computer, a desktop computer, a mobile computer, a laptop computer, a notebook computer, a terminal, a workstation, a server computer, a Personal Digital Assistant (PDA) device, a tablet computer, a network device, or any other suitable computing device.

In some embodiments, a system may include or may be, for example, a plurality of components that include a respective plurality of central processing units, e.g., a plurality of CPUs as described, a plurality of CPUs embedded in an on-board system or network, a plurality of chips, FPGAs or SOCs, microprocessors, transceivers, microcontrollers, a plurality of computer or network devices, any other suitable computing device, and/or any combination thereof. For example, a system as described herein may include one or more devices such as the control circuitry 1004.

FIG. 11 illustrates steps of an example method 1100 for controlling a light source of an endoscopic system according to the principles of the present disclosure. For example, one or more computing devices, processors or processing devices, etc. are configured to execute instructions to implement the method 1100, such as one or more of processors of the systems described herein. In an example, the computing system 1000 implements all or portions of the method 1100.

At 1104, the method 1100 receives an input corresponding to a command or instruction to supply light from a light source to an arthroscope. At 1108, the method 1100 activate safety light signal (e.g., activates an LED as described herein). In some examples, the light source may be activated at a same time as the safety light signal. In other examples, the light source may only be activated subsequent to step 1112 below.

At 1112, the method 1100 determines whether a light guide is properly coupled between a light port and the arthroscope. For example, the method 1100 determines whether an output of an optical receiver indicates that the safety light signal propagated from the light port, through the light guide, and back from an optical switch to an optical receiver of the light port. If true, the method 1100 continues to 1116. If false, the method 1100 continues to 1120.

At 1116, the method 1100 activates the light source and continues to 1112. In this manner, the method 1100 continues to determine whether the light guide is properly couple between the light port and the arthroscope during operation of the arthroscope (e.g., during a surgical procedure).

At 1120, the method 1100 deactivates (turns off) or reduces the intensity of the light source.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

The various steps and logic performed herein can be executed with non-volatile storage, memory, and processors. Non-volatile storage may include one or more persistent data storage devices such as a hard drive, optical drive, tape drive, non-volatile solid-state device, cloud storage or any other device configured to persistently store information. Processor may include one or more devices selected from high-performance computing (HPC) systems including high-performance cores, microprocessors, microcontrollers, digital signal processors, microcomputers, central processing units, field programmable gate arrays, programmable logic devices, state machines, logic circuits, analog circuits, digital circuits, or any other devices that manipulate signals (analog or digital) based on computer-executable instructions residing in memory. Memory may include a single memory device or a number of memory devices including, but not limited to, random access memory (RAM), volatile memory, non-volatile memory, static random access memory (SRAM), dynamic random access memory (DRAM), flash memory, cache memory, or any other device configured to store information.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the disclosure that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. As used herein, the phrases “at least one of A, B, and C” and “at least one of A, B, or C” should be construed to mean a logical (A OR B OR C), using a non-exclusive logical OR, and should not be construed to mean “at least one of A, at least one of B, and at least one of C.”

The terms “a,” “an,” “the,” and “said” as used herein in connection with any type of processing component configured to perform various functions may refer to one processing component configured to perform each and every function, or a plurality of processing components collectively configured to perform each of the various functions. By way of example, “A processor” configured to perform actions A, B, and C may refer to one or more processors configured to perform actions A, B, and C. In addition, “a processor” (or, “a processing device,” “a computing device,” and so on) configured to perform actions A, B, and C may also refer to a first processor configured to perform actions A and B, and a second processor configured to perform action C. Further, “A processor” configured to perform actions A, B, and C may also refer to a first processor configured to perform action A, a second processor configured to perform action B, and a third processor configured to perform action C.

In addition, in methods described herein where one or more steps are contingent upon one or more conditions having been met, it should be understood that the described method can be repeated in multiple repetitions so that over the course of the repetitions all of the conditions upon which steps in the method are contingent have been met in different repetitions of the method. For example, if a method requires performing a first step if a condition is satisfied, and a second step if the condition is not satisfied, then a person of ordinary skill would appreciate that the claimed steps are repeated until the condition has been both satisfied and not satisfied, in no particular order. Thus, a method described with one or more steps that are contingent upon one or more conditions having been met could be rewritten as a method that is repeated until each of the conditions described in the method has been met. This, however, is not required of system or computer readable medium claims where the system or computer readable medium contains instructions for performing the contingent operations based on the satisfaction of the corresponding one or more conditions and thus is capable of determining whether the contingency has or has not been satisfied without explicitly repeating steps of a method until all of the conditions upon which steps in the method are contingent have been met. A person having ordinary skill in the art would also understand that, similar to a method with contingent steps, a system or computer readable storage medium can repeat the steps of a method as many times as are needed to ensure that all of the contingent steps have been performed.

What is claimed is:

1. A method for operating a first light source of a surgical instrument, the method comprising:
selectively supplying from the first light source, illumination light to the surgical instrument via a light guide;
supplying, from a light source-side of the light guide and separate from the illumination light, a safety light via the light guide;
determining, at a computing device, whether the safety light is received at the light source-side of the light guide from an instrument-side of the light guide; and
in response to the determining, controlling the first light source to selectively deactivate or reduce an intensity of the illumination light.

2. The method of claim 1, wherein the surgical instrument is an arthroscope of an endoscopy system.

3. The method of claim 1, wherein the safety light is supplied from a second light source arranged in a light port of a console, and wherein the light guide is coupled to the light port.

4. The method of claim 3, wherein the console is an endoscopic console.

5. The method of claim 3, wherein the light port includes an optical receiver configured to receive the safety light, the method further comprising generating, using the optical receiver, an output indicating whether the safety light is received by the optical receiver.

6. The method of claim 5, wherein the instrument-side of the light guide includes an optical switch configured to receive the safety light from the second light source and supply the safety light to the optical receiver.

7. The method of claim 6, wherein the determining includes determining, based on the output, whether the safety light is received by the optical receiver, and wherein the controlling the first light source includes deactivating or reducing the intensity of the first light source in response to a determination that the optical receiver is not receiving the safety light.

8. The method of claim 6, further comprising supplying the safety light from the second light source to the optical switch using a first optical safety fiber and receiving the safety light at the optical receiver from the optical switch using a second optical safety fiber.

9. The method of claim 6, further comprising supplying the safety light from the second light source to the optical switch and receiving the safety light at the optical receiver from the optical switch using a first optical safety fiber.

10. A system for supplying illumination light to a surgical instrument, the system comprising:
a first light source, the first light source configured to supply the illumination light to the surgical instrument via a light port and a light guide coupled between the light port and the surgical instrument;
a second light source configured to supply, from a light source-side of the light guide and separate from the illumination light, a safety light via the light guide; and
a computing device configured to
determine whether the safety light is received at the light source-side of the light guide from an instrument-side of the light guide, and
in response to the determining, control the first light source to selectively deactivate or reduce an intensity of the illumination light.

11. The system of claim 10, wherein the surgical instrument is an arthroscope of an endoscopy system.

12. The system of claim 10, wherein the second light source is arranged in the light port.

13. The system of claim 12, wherein the light port is arranged in an endoscopic console.

14. The system of claim 12, wherein the light port includes an optical receiver configured to receive the safety light and generate an output indicating whether the safety light is received by the optical receiver.

15. The system of claim 14, wherein the instrument-side of the light guide includes an optical switch configured to receive the safety light from the second light source and supply the safety light to the optical receiver.

16. The system of claim 15, wherein the determining includes determining, based on the output, whether the safety light is received by the optical receiver, and wherein the controlling the light source includes deactivating or reducing the intensity of the first light source in response to a determination that the optical receiver is not receiving the safety light.

17. The system of claim 15, further comprising supplying the safety light from the second light source to the optical switch using a first optical safety fiber and receiving the safety light at the optical receiver from the optical switch using a second optical safety fiber.

18. The system of claim 15, further comprising supplying the safety light from the second light source to the optical switch and receiving the safety light at the optical receiver from the optical switch using a first optical safety fiber.

19. A surgical instrument, comprising:
a light post configured to couple the surgical instrument to a light guide to receive, from a light port, (i) illumination light for illuminating a surgical site and (ii) separate from the illumination light, safety light; and
an optical switch configured to receive the safety light and supply the safety light back to the light port via the light guide.

20. The surgical instrument of claim 19, wherein the surgical instrument is an arthroscope, and wherein the optical switch is arranged within the light post.
